(19) United States
(12) Patent Application Publication
(10) Pub. No.: US 2022/0117583 A1
(43) Pub. Date: Apr. 21, 2022

(54) Quantification of Dynamic Contrast Enhanced Imaging using Second Order Statistics and Perfusion Modeling

(71) Applicant: The Board of Trustees of the Leland Stanford Junior University, Stanford, CA (US)

(72) Inventors: Ahmed El Kaffas, San Jose, CA (US); Dimitre H. Hristov, Stanford, CA (US); Ashwin C. Reddy, Mineola, NY (US)

(21) Appl. No.: 17/504,350

(22) Filed: Oct. 18, 2021

Related U.S. Application Data
(60) Provisional application No. 63/092,721, filed on Oct. 16, 2020.

(51) Int. Cl. A61B 8/08; A61B 8/00

(52) U.S. Cl. CPC A61B 8/481; A61B 8/469

(57) ABSTRACT

A method for characterizing tissue, contrast agent behavior or microbubble behavior in dynamic contrast enhanced (DCE) medical image time-series data is provided. Time-series sequence of contrast enhanced medical imaging data is acquired during a contrast wash-in or a wash-out. Regions or volumes of interest (ROI/VOI) are selected and from those second order statistics is extracted at each frame of the time-series data. Each extracted second order statistic is assembled over time into a time-statistics curve (TSC). The TSC is normalized to emphasize a shape of the contrast behavior through the ROI or VOI instead of an intensity of the contrast behavior. The tissue, the contrast agent behavior, or the microbubble behavior is then characterized from the time-statistics curve.

### CROSS-REFERENCE TO RELATED APPLICATIONS

This application claims priority from U.S. Provisional Patent Application 63/092,721 filed Oct. 16, 2020, which is incorporated herein by reference.

### STATEMENT OF GOVERNMENT SPONSORED SUPPORT

This invention was made with Government support under contract CA195443 awarded by the National Institutes of Health. The Government has certain rights in the invention.

### FIELD OF THE INVENTION

This invention relates to methods to quantify contrast enhanced and molecular ultrasounds imaging.

### BACKGROUND OF THE INVENTION

Efficient, non-invasive, and powerful in-vivo molecular imaging is widely sought after for early screening and characterization of disease and treatment response in oncology. Additionally, many targeted therapeutic agents have been developed to combat disease that require ways to assess their effects in patients. Since the tumor vasculature is often a target of these therapeutic agents as well as involved in tumor progression, imaging tumor blood flow and acquiring parameters surrounding blood flow plays an important role in the treatment and monitoring of disease.

Contrast enhanced imaging is a valuable radiological imaging tool that can be used to assess tumor perfusion and its vasculature. The technique allows for the visualization of the dynamic perfusion of blood and allows for the quantification of several key hematological and morphological parameters in real time which can help inform clinicians about the lesion in question or the efficacy of treatment.

Contrast enhanced ultrasound (CEUS) imaging involves the injection of microbubbles, a contrast agent which allows to exclusively visualize the blood flow and blood volume in contrast-mode ultrasound images. The microbubble flow signal can be isolated from tissue signal and seen exclusively as the contrast agent travels through the vascular space in the lesion, thus providing key information on the vascular space.

A related imaging technique that has emerged in recent years, molecular contrast enhanced ultrasound (mCEUS) imaging, involves a combination of CEUS ultrasound imaging with the targeting of biomarkers in the lesion during imaging. Molecular ultrasound contrast agents are microbubbles that are targeted to specific molecular markers by adding high-affinity binding ligands that target specific molecular markers onto the surface of the microbubbles; this results in accumulation on the surface of endothelial cells expressing the target-receptor of interest at the lesion site, which results in signal only when the microbubbles bing to the endothelial cells. While CEUS imaging represents an unbound microbubble signal, mCEUS imaging represents a bound signal.

Contrast and Molecular ultrasound are of particular interest for imaging different disease attributes such as perfusion, vasculature and molecular expression, especially due to: high spatial and temporal resolution, real-time imaging, non-invasiveness, relatively low costs, lack of ionizing irradiation and wide availability of ultrasound systems. However, for both CEUS and mCEUS, there remains a need to effectively quantify signals and maximize on tissue specific information available through the temporal behavior of the contrast agent.

Traditionally, time-intensity curves (TICs) that measure average voxel intensity in a region-of-interest (ROI) of the image over time are used to quantify CEUS. However, quantification only using TICs could be missing some relevant features and parameters that provide information about disease status which could only be obtained through different quantification methods. In particular, a key assumption of the TIC is that perfusion is homogeneous, so heterogeneous perfusion cannot be characterized through a TIC approach alone. Thus, there is a need for quantification approaches that capture aspects of the tumor perfusion and vasculature based on the pattern of perfusion (not intensity focused), to highlight attributes of the underlying vascular system, and to account for heterogenous perfusions.

Similarly, in molecular ultrasound, one must usually wait at least 5 min or more after the molecular-contrast agent injection to be able to quantify bound from unbound signal, waiting for the unbound microbubbles to clear. Through this process, some signal is lost, and it poses a challenge for operators to image for up to 5 min. Thus, methods to extract features related to bound signal from unbound signal early on, based on the patterns of signal in the image, are highly sought-after.

Finally, both CEUS and mCEUS are heavily impacted by contrast agent handling at the time of injection (injection rate, microbubble concentration and consistency, activation time, syringe size and type, etc.) as well as operator handling of imaging system parameters and acquisition (signal gain, PRF, image FR, imaging angle, etc.). These inconsistencies in contrast-based ultrasound imaging make them difficult to quantify in a reliable manner, and ultimately only provide semi-quantitative estimates of perfusion or molecular signals.

### SUMMARY OF THE INVENTION

### Definitions

Dynamic contrast enhanced (DCE) medical imaging is defined as medical imaging examinations that involve capturing the temporal behavior of an injected contrast agent as it washes in and washes out a specific tissue of interest, through a cine or video acquisition. One can then characterize DCE cine through qualitative observations/features of the contrast behavior over time in disease, or qualitatively by modeling the signal intensities through a time intensity curve (TIC)
Texture features are second order statistical parameters used to capture interconnected voxel information, typically extracted on static images after building a grey-level co-concurrence matrix; these can include approaches that depend on grey-level co-occurrence matrix (GLMC; e.g. entropy, energy, contrast, homogeneity), wavelets, or can be based on custom features that relate the relationship of one pixel/voxel to another.
Time-statistics curves (TSC) including Time-texture curves (TTC) are determined which are defined as a time-series expression of a specific second-order statistical parameter, calculated within a ROI or a VOI at each frame of a Dynamic-Contrast Enhanced cine. This quantified parameter is assembled as a curve over time. These curves are often normalized to emphasize the pattern of perfusion and shape of the curve, instead of the intensities of the curve.
TSC measurements are statistics-based or modeling-based, parameters extracted from the TSC or TTC to summarize a specific property of tumor perfusion/vasculature (blood flow, blood volume, vascular architecture, etc.) or whether microbubbles are bound/unbound.

The present invention provides a computer-implemented method of characterizing or classifying tissues, contrast agent behavior, or microbubble behavior in dynamic contrast enhanced (DCE) medical image time-series data. A time-series sequence is acquired of contrast enhanced medical imaging data during a contrast wash-in or wash-out. A region of interest (ROI) or a volume of interest (VOI) that is to be characterized or classified for tissue or tissue properties. Second order statistics are extracted from within the ROI or VOI at each frame of a time series within a set period of the contrast wash-in or wash-out. Usually this is in the first 20-60 seconds of the wash-in. In one example, second-order statistics can be extracted in duplicate with altered extraction parameters (i.e. angle, vowel skip distance, etc.). In another example, second order statistics can be extracted under different binning/quantization condition to enable the full extent of possible grey-level resolutions. Second order statistics can include: co-occurrence matrix-based texture methods, wavelet methods, fast Fourier power spectrum methods, domain transform methods (transformation to laplace or other domains/dimensions), or other custom-based approaches that relate through parameterization one pixel-voxel to another (captures the interconnected nature of vascular systems through interconnected voxel analysis). The method continues by assembling each extracted second order statistic over time into a time-statistics curve (TSC, TTC, or the like). The assembled time-statistics curve is then used to characterize or classify tissue or tissue properties. The TSC is normalized to emphasize a shape of the contrast behavior through the ROI or VOI instead of an intensity of the contrast behavior.

In one example, one can perform model-based parameterization measurements of TSC or TTC. In another example, one can link back measurements to tissue properties of interest from histology or to contrast behavior. In yet another example, aggregate measurements from 1 or more TSC or TTC type can also be jointly fed into a feature-based machine learning model. In still another example, the TSC/TTC curves can be fed directly into a neural network to train a model that can either distinguish different tissue types or unique contrast behavior (i.e. bound from unbound microbubbles, without parametrization.

One specific application is in contrast ultrasound methods; there are two types of contrasts, targeted (i.e. molecular) and non-targeted. It is important that in targeted contrast enhanced ultrasound, one knows how much of the contrast actually targets or ‘attaches’ to endothelial cells, and to differentiate this targeted signal from non-targeted signals. Currently, one has to wait for more than 5 min to do so with steady hands holding the ultrasound probe over the lesion of interest, or use semi-invasive methods that manipulate the microbubbles and that may introduce bioeffects. Using second order statistics over time can help with this by looking at the relationship of one voxel to others nearby and determining if there is flow, or if the signal is ultimately changing the ‘texture’ of the image in manner similar to accumulation of the contrast through targeting.

Other applications in MRI, CT and Ultrasound are also available for quantification of 4D DCE methods.

Advantage of the method of the present invention is the fact it takes into account the nature of signal in a single voxel as a function of surrounding voxels, as opposed to taking that single voxel as an independent system. Embodiments provide a new way of looking at dynamic signals to provide new quantitative features to be used for perfusion analysis.

### BRIEF DESCRIPTION OF THE DRAWINGS

FIG. 1 shows a pipeline or computer-implemented method according to an exemplary embodiment of the invention.

FIG. 2 shows according to an exemplary embodiment of the invention representative TTC and TIC from a single 2D plane in the first 20 seconds in the cine.

FIG. 3 shows according to an exemplary embodiment of the invention a comparison of representative TICs longitudinally of bound and unbound cines from a control animal (a) and treated animal (b).

FIG. 4 shows according to an exemplary embodiment of the invention a comparison of representative GLCM difference entropy TTCs longitudinally of bound and unbound cines from a control animal (a) and treated animal (b).

FIG. 5. Average Pearson cross-correlations between bound and unbound curves for n=6 animals.

### DETAILED DESCRIPTION

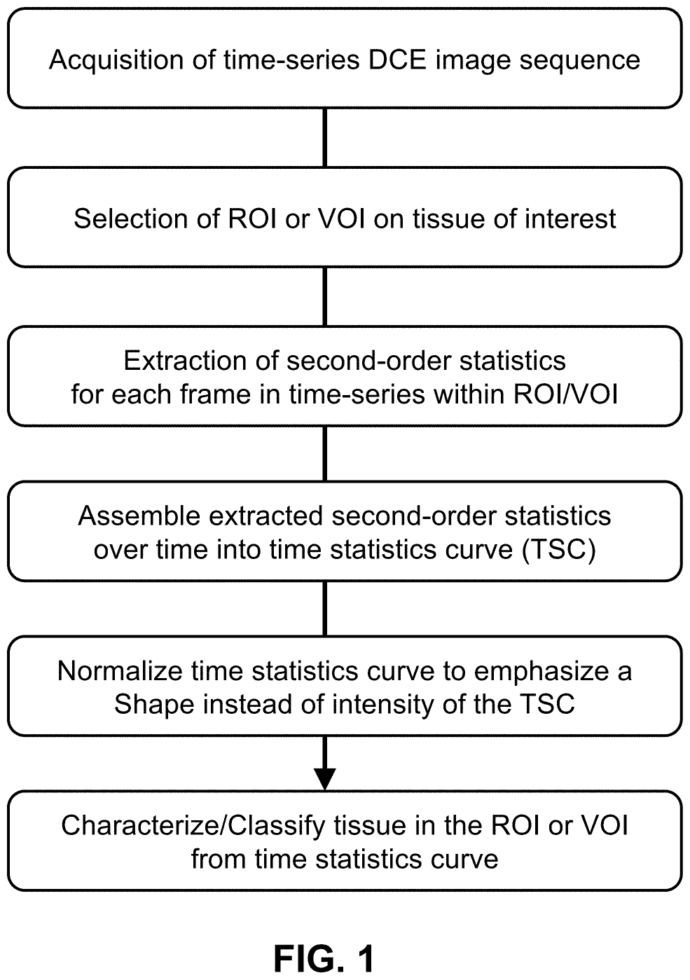

FIG. 1 shows an example of a pipeline for tissue characterization or classification using time-series data from medical images. This pipeline includes: acquiring a time-series sequence of contrast enhanced medical imaging data during a contrast wash-in, selecting a region of interest (ROI) or a volume of interest (VOI) that is to be characterized, extracting second order statistics within the ROI or the VOI at each time series within a set period of the contrast wash-in, assembling each extracted second order statistic over time into a time-statistics curve, and characterizing tissue from the time-statistics curve.

In another example, a pipeline for TSC or TTC extraction for data sets can include specific steps like: motion correction (this ensures that fames are aligned to minimize noise in the TSC), region/volume of interest (ROI/VOI) selection (operator would then highlight the tissue of interest such as tumor, in 2D or 3D, generating a mask that is applied to every frame/image in a DCE cine, within which the TSC will be computed), signal linearization (this steps allows to remove the effects of compression often applied to ultrasound images, by expanding the full dynamic range of a pixel/voxel), whole cine grey level quantization (this step aims to minimize the total number of grey-level values to reduce computation time and effectively generate a relationship of one voxel to another), frame-by-frame co-occurrence matrix and second-order statistics extraction, normalization of TTC (focus on the shape of the TTC curve instead of the intensities that may be skewed by non-standardized microbubble injections or operator-handling of acquisition parameters) and TTC modelling (this includes using statistical methods or modeling methods to parameterize each TTC of interest, extracting parameters related to specific tissue properties of interest based on a data-driven approach).

Noted is the importance of the idea of minimizing non-standardized handling of contrast agent at injection time, or even non-standardized acquisition parameters (ultrasound operators tend to play with a lot of buttons when imaging, which makes every scan look very different). TSCs or TTCs focus on the pattern of contrast flow, instead of intensity which is impacted by contrast-handling and operator.

Textures are one type of second order statistical features (see above for other types of second order features) in images that describe how voxels occur in patterns across space. Examples of texture features include Grey-Level Co-Occurrence Matrix (GLCM) features, which measure different attributes related to how two gray level voxels occur simultaneously within a certain distance of each other, and Neighborhood Grey Tone Difference Matrix (NGTDM) features, which measure the difference between a gray value and the average gray value of its neighbors. Instead of solely using TICs for quantification, computation of time-texture curves (TTCs), looking at textures in an ROI of the image over time, has potential to supplement first-order findings. These curves provide more information on vascular architecture and are more robust to differences in contrast agent injection or operator handling of imaging settings. Importantly, the voxel patterns that TTCs are based on are related to underlying vascular systems unlike the operator-dependent intensities that TICs utilize.

In one embodiment of the invention, the inventors aimed to evaluate dynamic contrast (unbound) and molecular (bound) microbubble signals. Using TTCs, the inventors attempted to achieve early differentiation between the bound and unbound signals and characterize heterogenous perfusion by going beyond measuring average intensity.

### Methods

A computational second-order statistics approach was used to quantify CEUS and mCEUS imaging. A total of 6 tumor bearing mice were imaged under an institutional approved protocol. All tumors were situated on the hind-leg and originated from colon cancer LS174T, a strain known to be responsive to treatment. Three mice received treatment for the tumor, and three mice served as controls and received no treatment. Once the tumors reached a volume of ˜100 mm³, they were imaged longitudinally on days 0, 1, 3, 7 and 10 of treatment using both Definity (Lantheus) and functionalized BR55 (Bracco) microbubbles, capturing both contrast and molecular modalities. The resulting 4D ultrasound cines were stored as a series of .raw image files. For the purposes of this invention, three time points were examined, the cines obtained on days 0, 3, and 10 for all mice. The first processing step involved conversion of these .raw files into the standard NIfTI format (.nii) using Python scripting, during which each cine was motion corrected and resampled to have their voxels be isotropic (0.3 cm/0.3 cm/0.3 cm).

The resampled videos were loaded into the ITK-SNAP software for ROI selection. 3D masks of the ROI were created by carefully annotating the cines from injection to washout in a 2D plane by hand to include as much of the tumor as possible while removing all surrounding tissue and interpolating the third axis.

The ultrasound data was loaded into another Python script for analysis. Here, additional pre-processing was done to linearize the signals. Additionally, grey-level quantization was performed with the whole cine (which was optimized at n=32 grey levels). The cines were masked, and initial TICs were generated to determine the frame in which the contrast agent washing-in period began. Frame-by-frame Grey-Level co-occurrence matrices (GLCM) and Neighborhood Grey Tone Difference Matrices (NGTDM) were calculated for the first 30 frames (roughly 20 seconds) following the beginning of the washing-in of contrast agent, resulting in extraction of 26 texture features in total. Average intensity of the ROI over the same period was also calculated.

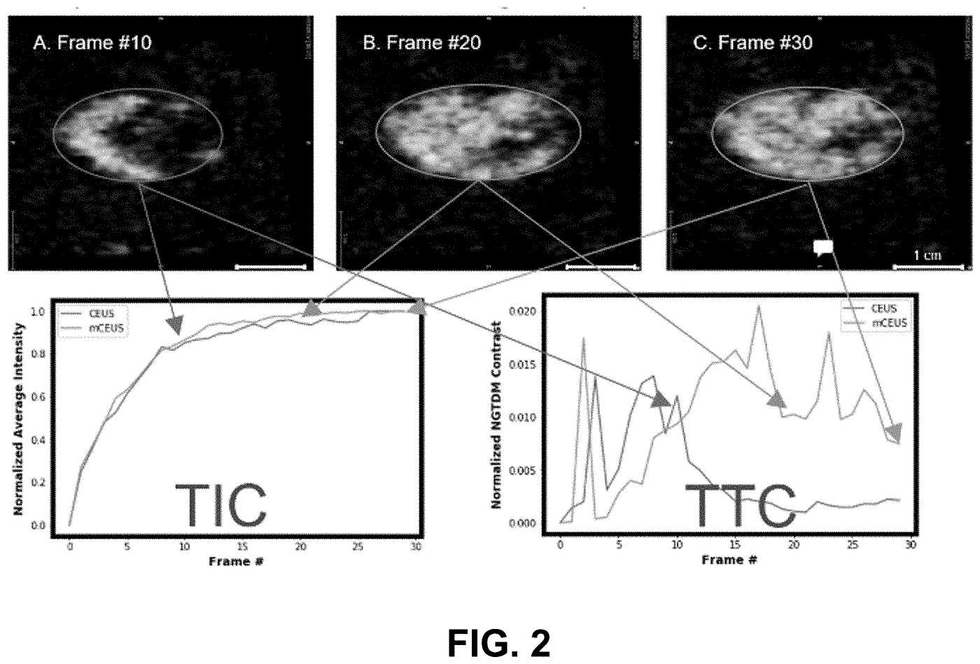

To model curves longitudinally and effectively differentiate bound and unbound signals, the extracted features were normalized for comparison. Finally, TICs and TTCs were generated and examined for each animal at each time point. Pearson cross-correlations were calculated for each pair of curves as a measure of synchrony for differentiation. The process of generating curves from the pre-segmented cines is shown in FIG. 2.

### Results

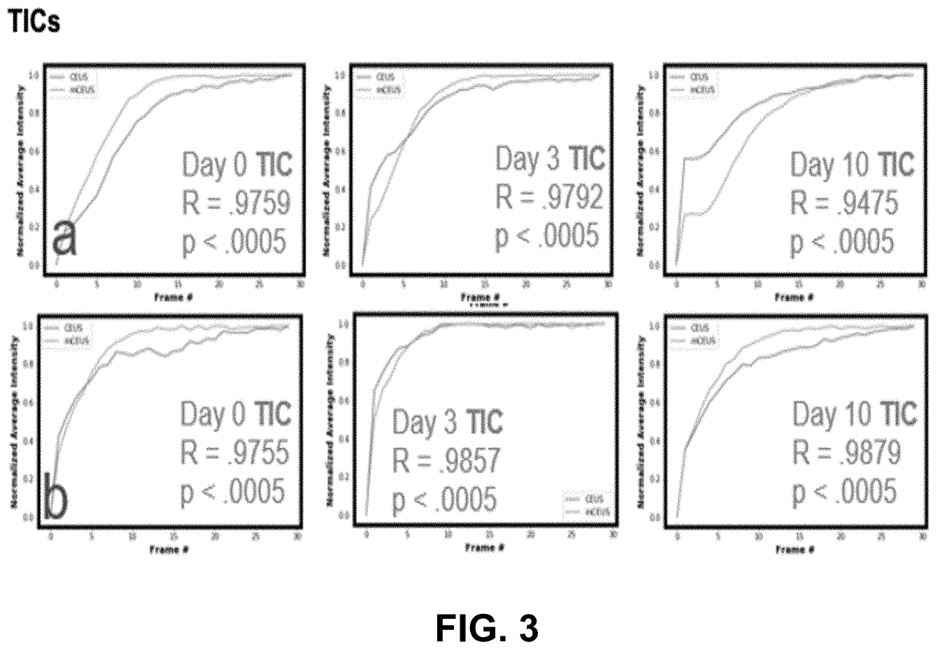

In a comparison of bound and unbound TICs from all the animals (FIG. 3), the shapes all appear to follow lognormal distributions. There is extremely highly significant synchrony between each pair of TICs (p<0.0005) which persists longitudinally as well. The shape of the TIC appears to be indistinguishable between bound and unbound signals.

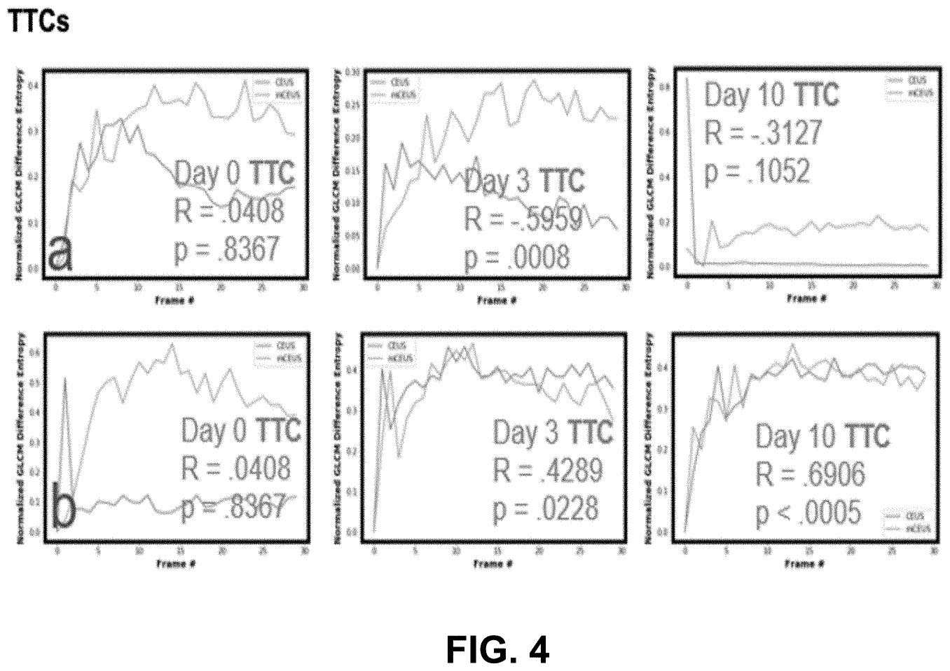

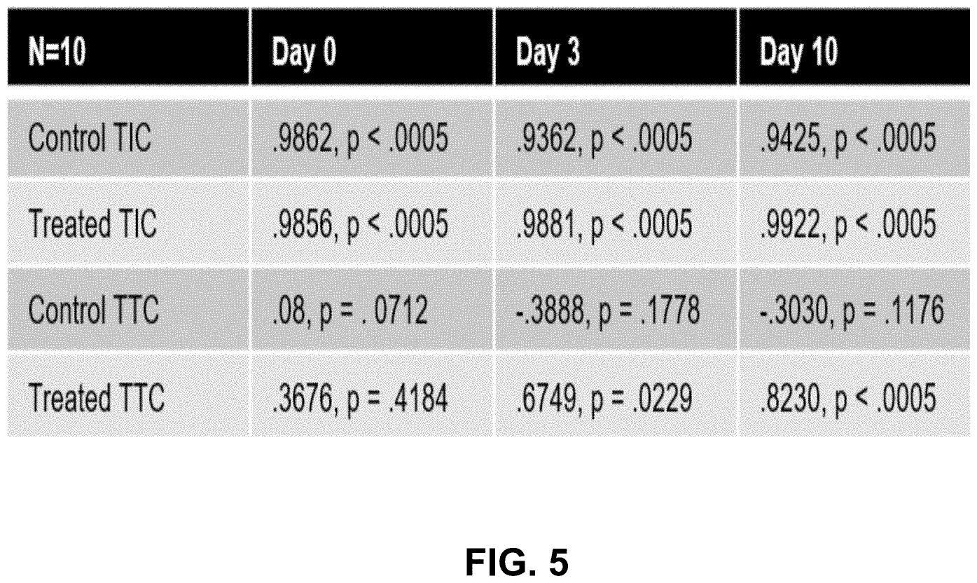

Comparing bound and unbound signals in TTCs (FIG. 4) across all 26 features resulted in varying degrees of differences. The three features that yielded the most reliably distinguishable curves from both the shapes of the TTCs and the Pearson cross-correlations were the GLCM Difference Entropy, NGTDM Busyness and NGTDM Contrast texture features. These TTCs did not yield an initial Pearson cross-correlation that indicated significant synchrony (p<0.05) and presented with distinct shapes as well. Another characteristic of the TTCs generated from these features is that, looking longitudinally, the TTCs of treated animals became more similar as the treatment progressed, both in their shape and in the Pearson cross-correlation. A summary of the compared synchrony statistics for GLCM Difference Entropy, a representative feature, is presented in FIG. 5.

When computing normalized TICs for CEUS and mCEUS signals, it was found that the resulting curves are essentially undifferentiable. They have the same shape (lognormal) and are synchronous with indistinguishable intensities. This pattern is observed across treated and untreated (control) tumors and in the same tumors over time. This supports the presence of and highlights some of the main issues with a TIC only approach, namely that TICs are too susceptible to differences in contrast agent injection and too dependent on operator-reliant intensities. While important information can be gleaned from TICs, there is clearly some other information, such as what is learned from a bound signal that cannot be learned from an unbound signal, that is being obscured by these limitations.

On the other hand, there appears to be significant promise behind a TTC approach. Unlike TICs, which, when normalized, could not differentiate bound and unbound signals, there were several texture features that yielded TTCs with the capability of differentiating the two signals within the first 20 seconds of contrast agent injection. Importantly, in control animals, the TTCs of these features maintained significant differences in the bound and unbound signals throughout the three time periods studied, suggesting a robustness to the measure. In treated animals however, as the course of treatment progresses, another clear trend arises: the signals become more similar. This likely reflects the decrease in binding that occurs in the mCEUS signal as fewer binding sites become available with less tumorous tissue, causing the bound mCEUS signal to resemble the unbound CEUS signal.

Of the three most effective texture features used to generate TTCs, two were NGTDM features. The first feature is busyness, which is a measure of the change from a voxel to its neighbor such that a high busyness indicates rapid changes of intensity, while the second feature is contrast, a measure of the spatial intensity change. The third feature came from the GLCM (difference entropy), and it represents the variability in neighborhood intensity value differences. It is interesting that these features produced desirable TTCs because they reflect some aspects of the heterogeneity of the voxels that they are working on. These features, and others, underscore one of the main benefits to TTC quantification over TIC quantification, which is the ability to characterize heterogenous perfusion rather than solely assuming homogenous perfusion.

The results indicate a use for texture analysis in the quantification of mCEUS, which can help better characterize a tumor's vasculature and provide more information about disease progression.

What is claimed is:

1. A method of characterizing tissue, contrast agent behavior or microbubble behavior in dynamic contrast enhanced (DCE) medical image time-series data, comprising:
(a) acquiring a time-series sequence of dynamic contrast enhanced medical imaging data during a contrast wash-in or a wash-out;
(b) selecting a region of interest (ROI) or a volume of interest (VOI) that is to be characterized;
(c) extracting second order statistics within the ROI or the VOI at each frame of a DCE time series within a set period of the contrast wash-in or wash-out;
(d) assembling each extracted second order statistic over time into a time-statistics curve (TSC);
(e) normalizing the TSC to emphasize a shape of the contrast behavior through the ROI or VOI instead of an intensity of the contrast behavior or the microbubble behavior; and
(f) characterizing the tissue, the contrast agent behavior, or the microbubble behavior from the time-statistics curve.
